(19) United States
(12) Patent Application Publication
(10) Pub. No.: US 2025/0051589 A1
(43) Pub. Date: Feb. 13, 2025

(54) COATING COMPOSITION FOR SILICONIZATION APPLICATION

(71) Applicants: DuPont Technology (Shanghai) Co., Ltd., Pudong New District (CN); DuPont Electronic Materials International, LLC., Marlborough, MA (US)

(72) Inventors: Xiaoman Qi, Shanghai (CN); Jian Lyu, Shanghai (CN); Chen-Yang Shiang, Taoyuan City (TW); Xiaogang Ma, Shanghai (CN)

(73) Assignees: DuPont Technology (Shanghai) Co., Ltd., Pudong New District, SH (CN); DuPont Electronic Materials International, LLC., Marlborough, MA (US)

(21) Appl. No.: 18/723,267

(22) PCT Filed: Dec. 21, 2022
(86) PCT No.: PCT/US2022/082129

§ 371 Date: Jun. 21, 2024

(30) Foreign Application Priority Data
Dec. 22, 2021 (CN) 202111577722.X

(51) Int. Cl. C09D 7/20; C09D 7/80; C09D 183/04; C09D 183/08

(52) U.S. Cl. CPC C09D 7/20; C09D 7/80; C09D 183/04; C09D 183/08

(57) ABSTRACT

Provided herein is an organic solvent to solve silicone polymer, to coat the silicone polymer on at least a portion of the surface of an article such as needles, syringes, stoppers and medicine bottles.

### CROSS-REFERENCE TO RELATED APPLICATION

This application claims priority to Chinese Patent App. No. 202111577722.X, filed on Dec. 22, 2021, which is hereby expressly incorporated herein in its entirety by reference.

### TECHNICAL FIELD

This disclosure relates to a coating composition used for coating of silicone materials on articles such as medical devices.

### BACKGROUND

Silicone materials are applied to a surface of many materials to lubricate these surfaces. Especially for medical apparatus, such silicone materials treatment (i.e. siliconization) is used because of the less chemical reactivity and high lubricating properties of silicone materials. Siliconization of medical apparatus also contributes decreasing pain or discomfort of patients. One example is that, when a needle of injection is penetrated the skin of a patient, the patient feels penetration force as a pain or discomfort. Such pain/discomfort is decreased when the friction of the needle surface is lower. Of course, siliconization on the surface of medical devices such as syringe barrel contributes smooth handling of these devices.

When silicone material is applied on a medical part, the silicone material is dissolved in an organic solvent, then the solution is coated on a surface of the medical part. After evaporation of the organic solvent, silicone material is coated on the surface of the medical part.

Many chlorofluorocarbons such as chlorofluorocarbon (CFC), hydrochlorofluorocarbons (HCFCs), and hydrofluorocarbon (HFC) have been used as a solvent to dissolve silicone materials because of these superior solubility and good properties in chemically/thermally stabilities. WO2011/007723A discloses a solvent for silicone compounds comprising HFC and 1-chloro-3,3,3-trifluoropropene.

However, due to ozone depletion potential (ODP) and global warming potential (GWP), CFC/HCFC/HFC type chlorofluorocarbons will be phased out by 2020 for developed countries, by 2030 for developing countries according to Montreal Declaration. Therefore, other candidates as a solvent of silicone materials are desired.

### BRIEF SUMMARY

This disclosure provides a coating composition comprising:
(i) about 90-about 99 wt % of organic solvent comprising:
   (a) about 25-about 50 wt % of hexamethyl disiloxane and
   (b) about 50-about 75 wt % of 1,1,1,4,4,4-hexafluoro-butene, and
(ii) about 1-about 10 wt % of silicone polymer.

This disclosure also provides an organic solvent to solve a silicone polymer represented by the formula (I);

(R¹R²R³)₃Si—O—[Si(R⁴)(R⁵)O]ₘ—[Si(R⁶)(R⁷)O]ₙ—Si(R⁸R⁹R¹⁰)₃ (I)
wherein R¹-R¹⁰ are respectively selected from alkyl group having 1 to 4 carbon atoms, H, OH, phenyl, vinyl, alkoxy, ON═C, CH₂(O)CHCH₂OCH₂CH₂CH₂, CH₂CH₂CH₂NH₂, CH₂CH₂CH₂NHCH₂CH₂NH₂ and OCOCH₃,
in which the organic solvent comprises:
   (a) about 25-about 50 wt % of hexamethyl disiloxane and
   (b) about 50-about 75 wt % of 1,1,1,4,4,4-hexafluoro-butene.

This disclosure further provides a method of forming a silicone polymer coating on at least a portion of the surface of an article, the method comprising the steps of:
(i) preparing an organic solvent comprising:
   (a) about 25-about 50 wt % of hexamethyl disiloxane and
   (b) about 50-about 75 wt % of 1,1,1,4,4,4-hexafluoro-butene,
(ii) mixing a silicone polymer and the organic solvent for about 1 to about 60 minutes to get a coating composition, then
(iii) applying the coating composition on a surface of the article.

### BRIEF DESCRIPTION OF THE DRAWINGS

The present disclosure will hereinafter be described in conjunction with the following drawing FIGURE, wherein

FIG. 1 shows a penetration test result for silicone coated needle.

### DETAILED DESCRIPTION

The following detailed description is merely exemplary in nature and is not intended to limit the current method or composition. Furthermore, there is no intention to be bound by any theory presented in the preceding background or the following detailed description. It is to be appreciated that all numerical values as provided herein, save for the actual examples, are approximate values with endpoints or particular values intended to be read as “about” or “approximately” the value as recited.

New organic solvents having excellent solubility to silicone materials are developed. The organic solvent is not chlorofluorocarbons so there is less concern about environmental problems.

Accordingly, one aspect of the disclosure is a coating composition comprising: (i) 90-99 wt % of organic solvent, comprising (a) 25-50 wt % of hexamethyl disiloxane and (b) 50-75 wt % of 1,1,1,4,4,4-hexafluoro-butene, and (ii) 1-10 wt % of silicone polymer.

Another aspect of the disclosure is an article having a silicone polymer coating on at least a portion of the surface of the article, in which the silicone polymer coating is formed from the solution.

Further aspect of the disclosure is an organic solvent to solve a silicone polymer represented by the formula (I);

(R¹R²R³)₃Si—O—[Si(R⁴)(R⁵)O]ₘ—[Si(R⁶)(R⁷)O]ₙ—Si(R⁸R⁹R¹⁰)₃ (I)
wherein R¹-R¹⁰ are respectively selected from alkyl group having 1 to 4 carbon atoms, H, OH, Phenyl, vinyl, alkoxy, ON═C, CH₂(O)CHCH₂OCH₂CH₂CH₂, CH₂CH₂CH₂NH₂, CH₂CH₂CH₂NHCH₂CH₂NH₂ and OCOCH₃, in which the organic solvent comprises (a) 25-50 wt % of hexamethyl disiloxane and (b) 50-75 wt % of 1,1,1,4,4,4-hexafluoro-butene.

Another aspect of the disclosure is a method to form a silicone polymer coating on at least a portion of the surface of the article, comprising the steps of:
(i) preparing an organic solvent comprising (a) 25-50 wt % of hexamethyl disiloxane and (b) 50-75 wt % of 1,1,1,4,4,4-hexafluoro-butene,
(ii) mixing the silicone polymer and the organic solvent for 1 to 60 minutes to get a coating composition, then
(iii) applying the coating composition on a surface of an article.

In the specification, the word ‘siliconization’ means a process to apply silicone materials on a surface of an article, to lubricate or hydrophobe the surface.

The organic solvent of this disclosure comprises 25-50 wt % of hexamethyl disiloxane (MM) and 50-75 wt % of 1,1,1,4,4,4-hexafluoro-butene (HFBE). The combination of these two shows excellent compatibility with silicone polymer. Preferably, the organic solvent comprises 30 to 40 wt % of MM and 70 to 60 wt % of HFBE.

The coating composition comprises, (i) 90-99 wt % of the organic solvent disclosed above, and (ii) 1-10 wt % of silicone polymer.

One example of the silicone polymer has the structure disclosed in formula (I).

(R¹R²R³)₃Si—O—[Si(R⁴)(R⁵)O]ₘ—[Si(R⁶)(R⁷)O]ₙ—Si(R⁸R⁹R¹⁰)₃ (I)

In the formula (I), R¹-R¹⁰ are respectively selected from alkyl group having 1 to 4 carbon atoms, H, OH, Phenyl, vinyl, alkoxy, ON═C, CH₂(O)CHCH₂OCH₂CH₂CH₂, CH₂CH₂CH₂NH₂, CH₂CH₂CH₂NHCH₂CH₂NH₂ and OCOCH₃. Preferably, R¹, R², R³ and R⁴ are methyl group and it is known as poly dimethyl siloxane (PDMS).

Examples of the silicone polymer disclosed as formula (I) include, Q7-9120 Silicone Fluid 20 cSt, Q7-9120 Silicone Fluid 100 cSt, Q7-9120 Silicone Fluid 350 cSt, Q7-9120 Silicone Fluid 500 cSt, Q7-9120 Silicone Fluid 1000 cSt, Q7-9120 Silicone Fluid 12500 cSt, Liveo 360 Medical Fluid 20 cSt, Liveo 360 Medical Fluid 100 cSt, Liveo 360 Medical Fluid 350 cSt, Liveo 360 Medical Fluid 1000 cSt, Liveo 360 Medical Fluid 12500 cst and MDX4-4159 50% Medical Grade Dispersion, those are silicone polymers available from DuPont.

The concentration of the silicone polymer in the coating composition is, 1 to 10 wt % based on the weight of the coating composition, preferably 1 to 6 wt % based on the weight of the coating composition, more preferably 2 to 6 wt % based on the weight of the coating composition.

### Materials and Articles

The coating composition of this disclosure can apply many materials, such as metals, plastics, rubbers, glass, papers, ceramic, latex and woods.

Many medical devices can have silicone polymer coatings. Examples of such medical devices include, needle, syringe, syringe barrel, urinary catheters, stoppers (rubber cover) and medicine bottle.

### Process

The method to form a silicone polymer coating on the surface of an article is disclosed below.

Silicone polymer and organic solvent comprising hexamethyl disiloxane (MM) and 1,1,1,4,4,4-hexafluoro-butene (HFBE) are mixed for 1 to 60 minutes to get a coating composition.

The coating composition is applied on the surface of an article. Any known method, such as splay coating, blade coating, painting, or dipping can be used. Exemplary, an article is dipped in the coating solution for 1 to 10 seconds, then blow off excess organic solvent for 1 to 5 seconds.

After volatilizing organic solvent, silicone polymer coating is formed on the surface of an article. Exemplary, an article is put for 1 to 20 minutes to volatilize organic solvent.

### Examples

### <Solubility Test>

Prepared a solvent comprising hexamethyl disiloxane (MM) and 1,1,1,4,4,4-hexafluoro-butene (HFBE) as disclosed in Table 1. Silicone polymer (Q7-9120 Silicone Fluid 12500 cSt) was mixed with the solvent to check the solubility of the solvent. The result is also shown in Table 1.

TABLE 1

| | Silicone | 30% MM | 33% MM | | |
|---|---|---|---|---|---|
| | polymer | and 70% | and 67% | Concentration | |
| No. | (g) | HFBE | HFBE | (% wt/wt) | Solubility |
| 1 | 0.325 | 4.012 | — | 7.5 | Good |
| 2 | 0.610 | — | 5.011 | 10.9 | Good |
| 3 | 0.610 | — | 4.005 | 13.2 | Bad |

### <Penetration Test>

Prepared an organic solvent comprising hexamethyl disiloxane (MM) and 1,1,1,4,4,4-hexafluoro-butene (HFBE) as the weight ratio of 33:67. Mixed silicone polymer (Liveo 360 Medical Fluid-12500 cSt or MDX4-4159 50% Medical Grade Dispersion) in the organic solvent, then stirred for 1 to 60 minutes to get a coating composition. The concentration of the silicone polymer in the organic solution was 6 wt %.

Stainless needle with O.D. 1.65 mm, and TPU membrane film with 0.4 mm thickness were used. Five (5) stainless needles were dipped into the coating solution for 1 to 5 seconds, then blew off excess solution for 1 to 5 seconds. Then the needles were put in the oven at 50 degrees C. for 10 minutes, to form a coating on the surface of the needles.

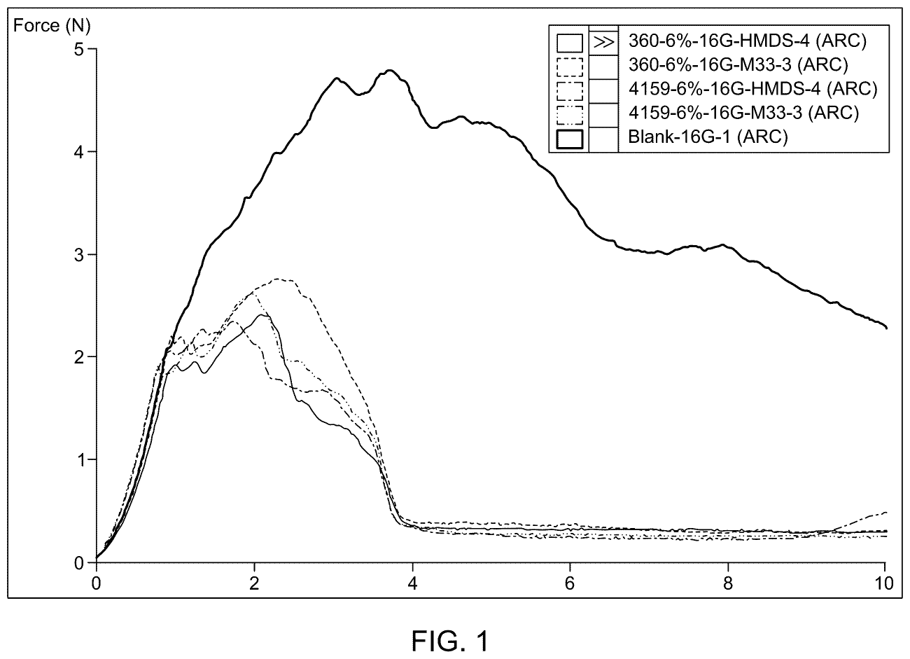

Measured penetration forces by designed time interval. Took average of 5 needles (n=5) and recorded in Table 1 to represent treatment performance. FIG. 1 also shows the penetration test result.

Texture Analyser was used for penetration test.

### Meanings of F0, F1, F2 and F3

F0: Force for passing the needle tip (piercing force)

F1: Force for cutting with the needle cutting edges (cutting force)

F2: Force for dilatation of the cut from the needle primary bevel (dilatation force)

F3: Force for sliding of the needle tube (sliding force)

TABLE 1

| Test ID | F0/N | F1/N | F2/N | F3/N |
|---|---|---|---|---|
| 360-12500 (6%)-16G-HMDS | 2.233 | 2.773 | 2.436 | 0.41 |
| 360-12500 (6%)-16G-M33 | 2.202 | 2.799 | 2.701 | 0.383 |
| 4159 (6%)-16G-HMDS | 2.028 | 2.407 | 1.924 | 0.284 |
| 4159 (6%)-16G-M33 | 2.244 | 2.439 | 1.856 | 0.287 |
| Blank | 2.166 | 3.995 | 4.723 | 3.608 |

The penetration performance is very similar between HDMS and the organic solvent and better than blank (no treatment), so the new organic solvent is good solvent replacement for siliconization application.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration in any way. Rather, the foregoing detailed description will provide those skilled in the art with a convenient road map for implementing an exemplary embodiment. It being understood that various changes may be made in the function and arrangement of elements described in an exemplary embodiment without departing from the scope as set forth in the appended claims.

What is claimed is:

1. A coating composition comprising:
(i) about 90-about 99 wt % of an organic solvent comprising:
(a) about 25-about 50 wt % of hexamethyl disiloxane and
(b) about 50-about 75 wt % of 1,1,1,4,4,4-hexafluoro-butene, and
(ii) about 1-about 10 wt % of a silicone polymer.

2. The solution of claim 1, wherein the silicone polymer is represented by the formula (I):
(R¹R²R³)₃Si—O—[Si(R⁴)(R⁵)O]ₘ—[Si(R⁶)(R⁷)O]ₙ—Si(R⁸R⁹R¹⁰)₃ (I)
wherein R¹-R¹⁰ are respectively selected from an alkyl group having 1 to 4 carbon atoms, H, OH, phenyl, vinyl, alkoxy, ON═C, CH₂(O)CHCH₂OCH₂CH₂CH₂, CH₂CH₂CH₂NH₂, CH₂CH₂CH₂NHCH₂CH₂NH₂ and OCOCH₃.

3. The solution of claim 1, wherein the silicone polymer is selected from polydimethylsiloxane and amino-alkoxy dimethylsiloxane.

4. An article having a silicone polymer coating on at least a portion of the surface of the article, in which the silicone polymer coating is formed from the coating composition of claim 1.

5. The article of claim 4, wherein the thickness of the silicone polymer coating is about 0.01 to about 1 micrometer.

6. The article of claim 4, wherein the article comprises a material selected from metals, plastics, rubbers, glass, papers, ceramic, latex and woods.

7. The article of claim 4, wherein the article is a medical device.

8. The article of claim 7, wherein the medical device is selected from a needle, a syringe, a stopper, and a medicine bottle.

9. An organic solvent to solve a silicone polymer represented by the formula (I);
(R¹R²R³)₃Si—O—[Si(R⁴)(R⁵)O]ₘ—[Si(R⁶)(R⁷)O]ₙ—Si(R⁸R⁹R¹⁰)₃ (I)
wherein R¹-R¹⁰ are respectively selected from alkyl group having 1 to 4 carbon atoms, H, OH, phenyl, vinyl, alkoxy, ON═C, CH₂(O)CHCH₂OCH₂CH₂CH₂, CH₂CH₂CH₂NH₂, CH₂CH₂CH₂NHCH₂CH₂NH₂ and OCOCH₃,
in which the organic solvent comprises:
(a) about 25-about 50 wt % of hexamethyl disiloxane and
(b) about 50-about 75 wt % of 1,1,1,4,4,4-hexafluoro-butene.

10. A method of forming a silicone polymer coating on at least a portion of a surface of an article, the method comprising the steps of:
(i) preparing an organic solvent comprising:
(a) about 25-about 50 wt % of hexamethyl disiloxane and
(b) about 50-about 75 wt % of 1,1,1,4,4,4-hexafluoro-butene,
(ii) mixing a silicone polymer and the organic solvent for about 1 to about 60 minutes to get a coating composition, then
(iii) applying the coating composition on the surface of the article.
